(19) United States
(12) Patent Application Publication
(10) Pub. No.: US 2024/0047069 A1
(43) Pub. Date: Feb. 8, 2024

(54) METHODS AND APPARATUSES FOR DETERMINING STATE INFORMATION AND HEALTH INDEX INFORMATION

(71) Applicant: BOE Technology Group Co., Ltd., Beijing (CN)

(72) Inventors: Fang ZHAI, Beijing (CN); Yanyang HU, Beijing (CN); Fuchen TIAN, Beijing (CN); Yubin ZHOU, Beijing (CN)

(21) Appl. No.: 17/639,270

(22) PCT Filed: Apr. 30, 2021
(86) PCT No.: PCT/CN2021/091616

§ 371 Date: Nov. 3, 2022

(51) Int. Cl. G16H 50/30; G16H 50/70

(52) U.S. Cl. CPC G16H 50/30; G16H 50/70

(57) ABSTRACT

The present disclosure provides a method of determining state information. The method includes: obtaining first-type state information of a first user; according to the first-type state information of the first user and previously-stored first-type state information of at least one second user, determining a comparable user for the first user from the at least one second user; according to second-type state information of the comparable user, determining second-type state information of the first user.

### TECHNICAL FIELD

The present disclosure relates to the field of data processing, and in particular to a method of determining state information, a method of determining health index information, an apparatus for determining state information, an apparatus for determining health index information, a composition analysis system, an electronic device and a computer readable storage medium.

### BACKGROUND

When a user performs human body composition analysis, the user needs to input state information, and may determine information of some compositions in the body of the user based on the state information inputted. In the state information to be input by the user, some state information, such as age, and gender of the user, can be determined by the user relatively accurately, while some state information cannot be determined by the user accurately.

### SUMMARY

Some embodiments of the present disclosure provide a method of determining state information, a method of determining health index information, an apparatus for determining state information, an apparatus for determining health index information, a composition analysis system, an electronic device and a computer readable storage medium.

According to some aspects of embodiments of the present disclosure, there is provided a method of determining state information. The method includes: obtaining first-type state information of a first user; according to the first-type state information of the first user and previously-stored first-type state information of at least one second user, determining a comparable user for the first user from the at least one second user; according to second-type state information of the comparable user, determining second-type state information of the first user.

In some embodiments, according to the first-type state information of the first user and the previously-stored first-type state information of at least one second user, determining the comparable user for the first user from the at least one second user includes: determining a first feature vector according to the first-type state information of the first user, and determining a second feature vector according to the first-type state information of the second user; calculating a similarity between the first feature vector and the second feature vector, and determining a second user with a similarity larger than a similarity threshold as the comparable user.

In some embodiments, the first feature vector X includes elements of n dimensions, and the elements in the first feature vector X correspond to the first-type state information, wherein the element of an i-th dimension is denoted as xi; the second feature vector Y includes elements of n dimensions, and the elements in the second feature vector Y correspond to the second-type state information, wherein the element of an i-th dimension is denoted as yi, n is an integer equal to or larger than 1, 1≤i≤n; calculating the similarity between the first feature vector and the second feature vector includes: calculating the similarity γ(X,Y) between the first feature vector X and the second feature vector Y based on the following formula:

$$γ ( X , Y ) = \frac{n Σ {x}_{i} {y}_{i} - Σ {x}_{i} Σ {y}_{i}}{\sqrt{n Σ {x}_{i}^{2} - {( Σ {x}_{i} )}^{2}} * \sqrt{n Σ {y}_{i}^{2} - {( Σ {y}_{i} )}^{2}}}$$

Where the first feature vector X includes elements of n dimensions, and the second feature vector Y includes elements of n dimensions, xi is an element of an i-th dimension in the first feature vector X, yi is an element of an i-th dimension in the second feature vector Y, n is an integer equal to or larger than 1, 1≤i≤n.

In some embodiments, determining the comparable user for the first user from the at least one second user includes: ranking the second users with similarities larger than the similarity threshold based on a descending order of similarities; determining a second user with a rank prior to a first order threshold as the comparable user.

In some embodiments, determining the comparable user for the first user from the at least one second user includes: determining a first feature vector according to the first-type state information of the first user, and determining a second feature vector according to the first-type state information of the second user; calculating a similarity between the first feature vector and the second feature vector, and ranking the second users according to a descending order of similarities; determining a second user with a rank prior to a second order threshold as the comparable user.

In some embodiments, according to the second-type state information of the comparable user, determining the second-type state information of the first user includes: calculating a mean value of the second-type state information of all comparable users as the second-type state information of the first user.

In some embodiments, determining, from second-type state information of the comparable users, second-type state information of a comparable user which has been confirmed by the corresponding comparable user as target state information, and determining the corresponding comparable user of the target state information as a target user; calculating a mean value of the target state information belonging to the same target user as a state-information mean value of the target user; according to a similarity between the target user and the first user along with the state-information mean value of the target user, determining the second-type state information of the first user.

In some embodiments, the second-type state information of the first user is determined based on the following formula:

$$v ( a , p ) = \frac{{Σ}_{b ∈ S ( a , I ) ⋂ N ( p )} {l}_{ab} \overline{{d}_{bp}}}{{Σ}_{b ∈ S ( a , U ) ⋂ N ( p )} {l}_{ab}} ;$$

where, S(a,U) represents a set of comparable users U of a first user a, N(p) represents a set of target users, l_{ab} represents a similarity between the first user a and the target user b, and d̅_̅{̅b̅p̅}̅ represents the above state-information mean value of the target user b.

In some embodiments, the method of determining state information is applicable to a terminal.

In some embodiments, the first-type state information includes at least one of: gender information, age information, body height information, body weight information, position information, time information and occupation information.

In some embodiments, the second-type state information includes at least one of: tare weight information, blood pressure information, pulse information, and heart beat information.

According to some aspects of embodiments of the present disclosure, there is provided a method of determining health index information. The method includes: according to the first-type state information and the second-type state information of the first user in the above method of determining state information, determining body composition information of the first user.

In some embodiments, the method further includes: providing the second-type state information of the first user to the first user.

In some embodiments, when receiving a modification instruction for the second-type state information of the first user, determining the health index information of the first user according to modified second-type state information of the first user.

In some embodiments, the method is applicable to a health detection device.

According to some aspects of embodiments of the present disclosure, there is provided a composition analysis system, including a terminal and a health detection device; the terminal is configured to: obtain first-type state information of a first user; according to the first-type state information of the first user and previously-stored first-type state information of at least one second user, determine a comparable user for the first user from the at least one second user; according to second-type state information of the comparable user, determine second-type state information of the first user; transmit the second-type state information of the first user to the health detection device; the health detection device is configured to: based on the first-type state information and the second-type state information of the first user, determine the health index information of the first user.

In some embodiments, the terminal is configured to determine, from second-type state information of the comparable users, second-type state information of a comparable user which has been confirmed by the corresponding comparable user as target state information, and determine the corresponding comparable user of the target state information as a target user; calculate a mean value of the target state information belonging to the same target user as a state-information mean value of the target user; according to a similarity between the target user and the first user along with the state-information mean value of the target user, determine the second-type state information of the first user.

In some embodiments, the health detection device is further configured to: provide the second-type state information of the second user to the second user, and when receiving a confirmation instruction of the second user for the second-type state information of the second user, determine the second-type state information of the second user is the target state information confirmed by the second user.

In some embodiments, the terminal is configured to: according to the target state information confirmed by the second user obtained from the health detection device, determine, from second-type state information of the comparable users, second-type state information of a comparable user which has been confirmed by the corresponding comparable user as target state information, and determine the corresponding comparable user of the target state information as a target user.

In some embodiments, the health detection device is further configured to provide the second-type state information of the first user to the first user.

In some embodiments, the health detection device is further configured to, when receiving a modification instruction for the second-type state information of the first user, determine the health index information of the first user according to modified second-type state information of the first user.

In some embodiments, the terminal is configured to, according to information input by the first user, determine at least one of the following first-type state information of the first user: gender information, age information, body height information, body weight information, position information, time information and occupation information.

In some embodiments, the second-type state information includes at least one of: tare weight information, blood pressure information, pulse information, and heart beat information.

According to some aspects of embodiments of the present disclosure, there is provided an apparatus for determining state information. The apparatus includes: a state obtaining module, configured to obtain first-type state information of a first user; a similarity determining module, configured to according to the first-type state information of the first user and previously-stored first-type state information of at least one second user, determine a comparable user for the first user from the at least one second user; a state determining module, configured to, according to second-type state information of the comparable user, determine second-type state information of the first user.

According to some aspects of embodiments of the present disclosure, there is provided an apparatus for determining health index information. The apparatus includes: a composition analyzing module, configured to, according to the first-type state information and the second-type state information of the first user in the above method of determining state information, determine body composition information of the first user.

According to some aspects of embodiments of the present disclosure, there is provided an electronic device, including a processor and a memory configured to store instructions executable by the processor, where the processor is configured to implement the above method of determining state information, and/or the above method of determining health index information.

According to some aspects of embodiments of the present disclosure, there is provided a computer readable storage medium storing computer programs thereon, where the programs are executed by a processor to implement the above method of determining state information, and/or the above method of determining health index information.

### BRIEF DESCRIPTION OF THE DRAWINGS

To describe the technical solutions in the embodiments of the present disclosure more clearly, drawings required in descriptions of the examples of the present disclosure will be briefly introduced below. It is apparent that the drawings described below are merely embodiments of the present disclosure and other drawings may be obtained by those of ordinary skill in the prior art based on these drawings in the embodiments of the present disclosure.

FIG. 1 is a flowchart illustrating a method of determining state information according to some embodiments of the present disclosure.

FIG. 2 is a flowchart illustrating a method of determining state information according to some embodiments of the present disclosure.

FIG. 3A is a flowchart illustrating a method of determining state information according to some embodiments of the present disclosure.

FIG. 3B is a flowchart illustrating a method of determining state information according to some embodiments of the present disclosure.

FIG. 4 is a flowchart illustrating a method of determining state information according to some embodiments of the present disclosure.

FIG. 5 is a flowchart illustrating a method of determining state information according to some embodiments of the present disclosure.

FIG. 6 is a flowchart illustrating a method of determining health index information according to some embodiments of the present disclosure.

FIG. 7 is a schematic diagram illustrating a composition analysis system according to some embodiments of the present disclosure.

FIG. 8 is a block diagram illustrating an apparatus for determining state information according to some embodiments of the present disclosure.

FIG. 9 is a block diagram illustrating an apparatus for determining health index information according to some embodiments of the present disclosure.

FIG. 10 is a block diagram illustrating an apparatus for determining state information according to some embodiments of the present disclosure.

### DETAILED DESCRIPTION OF THE EMBODIMENTS

The technical solutions of the embodiments of the present disclosure will be fully and clearly described below in combination with the accompanying drawings in the embodiments of the present disclosure. Apparently, the embodiments described herein are merely some embodiments of the present disclosure rather than all embodiments. Other embodiments obtained by those skilled in the art based on these embodiments without making creative work shall all fall within the scope of protection of the present disclosure.

In the related arts, for those state information that cannot be determined by a user accurately, the user needs to input a value of the state information based on subjective guess and the value guessed by the user subjectively is usually low in accuracy. Thus, it is difficult to analyze body composition of the user accurately based on the state information input by the user. In an application scenario of health-care hut, after personal information is input by a work station, the user needs to go to a detection device to input another part of user parameters, bringing troublesome use and tedious operations.

FIG. 1 is a flowchart illustrating a method of determining state information according to some embodiments of the present disclosure. The method shown in the embodiments is applicable to an electronic device such as a terminal provided for a user to input state information. The terminal includes but not limited to smart phone, tablet computer, personal computer, wearable device and work station and the like.

As shown in FIG. 1, the method of determining state information includes the following steps.

At step S101, first-type state information of a first user is obtained.

At step S102, according to the first-type state information of the first user and previously-stored first-type state information of at least one second user, a comparable user for the first user is determined from the at least one second user.

At step S103, according to second-type state information of the comparable user, second-type state information of the first user is determined.

In some embodiments, the first user may input the first-type state information to the terminal and thus the terminal obtains the first-type state information of the first user. Implementation of the first user inputting the first-type state information includes but not limited to: touch input, voice input, direct measurement and the like. For example, the terminal is a work station of a health-care hut, and the first user may input the first-type state information through the work station.

In an application scenario of a health-care hut, at least one work station and at least one type of detection devices are disposed in the hut and each type of detection devices may include a plurality of devices. The user may log onto the work station and input the first user information. The user may select a device to be used through the work station, and the work station may perform detection guidance to the user in the form of user interaction interface etc. The user may, according to the detection guidance provided by the work station, move to the position of the corresponding detection device for corresponding detection. After the user is detected by the detection device, the corresponding detection result will be sent to the work station for displaying. The health-care hut may be disposed in a place such as a community and thus residents in the community may go into the hut to perform health detection by a device, thereby helping the residents to perform health self-management.

In some embodiments, the first-type state information may be state information which can be accurately determined by the user, for example, may include at least one of: gender information, age information, body height information, body weight information, position information, time information, season information, occupation information, people classification information (e.g. ordinary person, athlete). For example, the work station of the health-care hut provides a user interaction interface to display and prompt the user to input at least one of: gender information, age information, body height information, body weight information, occupation information, people classification information (e.g. ordinary person, athlete), and the user may perform input through the user interaction interface. For example, the work station of the health-care hut may obtain local geographical location information through automatic positioning and automatically obtain local time information and season information through the work station system. For example, part of the first-type state information may be stored after the first use of the user and will not be re-input in next use but automatically obtained based on login information. The user may also modify part of the first-type state information as needed and then store the modified information. For example, the invariable first-type state information such as gender will not be changed again after first input, and the first-type state information such as age which may change based on a specific rule may be updated based on the specific rule after the first input, whereas the information such as body weight and occupation etc. may change in subsequent use and thus may be modified as needed. In some embodiments, the second-type state information may be state information which generally cannot be determined by the user accurately, for example, may include at least one of tare weight information, blood pressure information, pulse information, and heart beat information. The tare weight information refers to a weight of other articles than the body of the user, which may include but not limited to clothing, shoes, mobile phone, and jewelry and the like.

In some embodiments, the terminal may store first-type state information and second-type state information of at least one second user in advance, for example, may determine a large number of second users as samples in advance, and then measures/inputs their first-type state information and second-type state information, thus obtaining accurate first-type state information and second-type state information of the second user.

In some embodiments, the first-type state information of the first user and the second-type state information determined by the first user may be stored as samples for subsequent use by the user.

In some embodiments, the first-type state information of the first user and the second-type state information determined by the first user may be subsequently added to previously-stored samples, so as to expand the samples. The second-type state information determined by the first user refers to the second-type state information confirmed by the first user after the second-type state information is determined for the first user based on the embodiment shown in FIG. 1 and pushed to the first user. This part of second-type state information is confirmed by the user and thus is accurate and may be stored as valid samples.

According to some embodiments of the present disclosure, after the first-type state information of the first user is obtained, a comparable user for the first user, for example, a second user with a similarity with the first user larger than a similarity threshold, may be determined from the second users according to the first-type state information of the second user and the first-type state information of the first user. If the first-type state information of the comparable user is similar to the state information of the first user, it is highly possible that the second-type state information of the comparable user is similar to the state information of the first user. Therefore, according to the second-type state information of the comparable user, the second-type state information of the first user may be determined.

Thus, it is not required for the first user to determine the second-type state information by subjective guess but determine a comparable user for the first user based on a large number of samples and further predict the second-type state information of the first user based on the second-type state information of the comparable user, thus facilitating improving the accuracy of determining the second-type state information, and further helping perform accurate health index analysis for the first user based on the second-type state information subsequently.

It is noted that, one or more comparable users may be determined. In a case of determining one comparable user, the second-type state information of the comparable user may be pushed as the second-type state information of the first user to the first user; in a case of determining a plurality of comparable users, the second-type state information of the first user may be determined based on the second-type state information of the plurality of comparable users, for example, with a similarity between the first user and the comparable user as a weight value, a weighted sum may be calculated for the second-type state information of the plurality of comparable users to obtain a result as the second-type state information of the first user. For another example, the second-type state information of the comparable user with highest similarity is taken as the second-type state information of the first user.

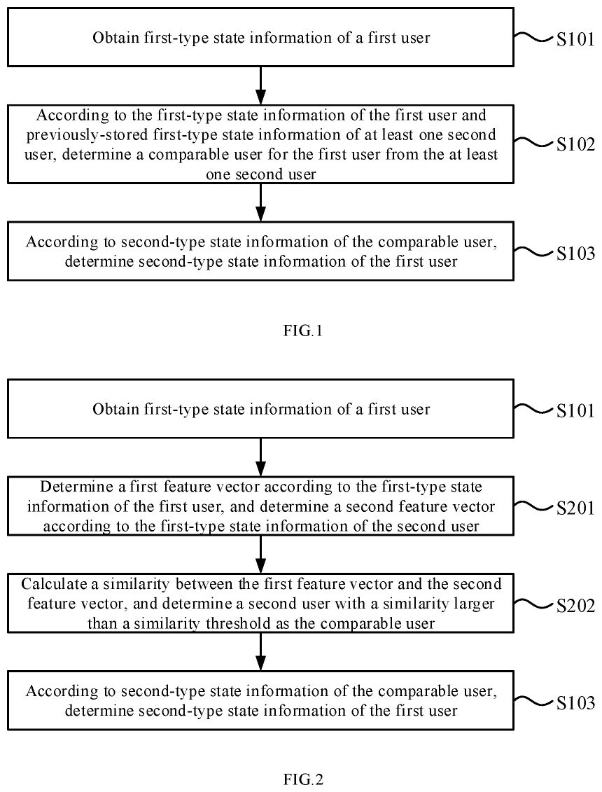

FIG. 2 is a flowchart illustrating a method of determining state information according to some embodiments of the present disclosure. As shown in FIG. 2, according to the first-type state information of the first user and the previously-stored first-type state information of at least one second user, determining a comparable user for the first user from the at least one second user includes the following steps.

At step S201, a first feature vector is determined according to the first-type state information of the first user, and a second feature vector is determined according to the first-type state information of the second user.

At step S202, a similarity between the first feature vector and the second feature vector is calculated, and a second user with a similarity larger than a similarity threshold is determined as the comparable user.

In some embodiments, in order to determine a similarity between the second user and the first user, a first feature vector may be determined according to the first-type state information of the first user, and a second feature vector may be determined according to the first-type state information of the second user, such that the first feature vector represents the first user and the second feature vector represents the second user. Further, a similarity between the first feature vector and the second feature vector may be calculated as a similarity between the first user and the second user, so as to determine a second user with a similarity larger than a similarity threshold as the comparable user.

In some embodiments, a similarity threshold may be set, such that a second user with a similarity larger than the similarity threshold may be determined as the comparable user. For example, the similarity threshold may be set as needed, which is not limited herein. For example, when the similarity threshold is set to 0.5, a second user with a similarity larger than 0.5 may be determined as the comparable user.

A number of pieces of the first-type state information of the first user may be n, and a number of pieces of the first-type state information of the second user may also be n. In this case, the first feature vector X may be a n-dimension vector including n pieces of first-type state information xᵢ of the first user and correspondingly, the second feature vector Y may be a n-dimension vector including n pieces of first-type state information yᵢ of the second user, where n is an integer equal to or larger than 1, and 1≤i≤n.

For example, there are five pieces of first-type state information: gender information, age information, body height information, body weight information, and time information.

The gender information may represent female with 0 and male with 1; the age information, the body height information, the body weight information may be corresponding values, for example, age 28 corresponds to the age information 28, body height 174 cm corresponds to the body height information 174, and body weight 50 kg corresponds to the body weight information 50; the time information may represent 6-9 o'clock with 1, and 9-12 o'clock with 2, 12-15 o'clock with 3, and 15-18 o'clock with 4.

For example, the first-type state information of the first user is female, age 25, height 167 cm and weight 50 kg, and a time that the first user inputs the first-type state information is 14 o'clock. In this case, the first feature vector generated based on these first-type state information may be represented as (0, 25, 167, 50, 14). Similarly, for the first-type state information of the second user, the second feature vector may also be generated in the above implementation, which is not further described herein.

In some embodiments, the first-type state information and the second-type state information may be converted to feature vectors in the form of one-hot code, word2vec or TransE or the like.

In some embodiments, the number of pieces of the first-type state information of the first user may be different from the number of pieces of the first-type state information of the second user as long as the similarity therebetween can be calculated. For example, if the number of pieces of the first-type state information of the first user is different from that of the second user, the missing value(s) may be supplemented as null to make the numbers of the first user and the second user to be the same, and the position of the corresponding vector element is set to 0, thus the first feature vector and the second feature vector may be of the same number of dimensions.

In some embodiments, the similarity between the first feature vector and the second feature vector may be determined by calculating an included angle between the first feature vector and the second feature vector. For example, a distance therebetween may be calculated by calculating their dot product or cosine value and the like, or determined in the following implementation. But no limitation is made to the two implementations and the specific implementation may be selected as needed.

In some embodiments, the first feature vector X includes elements of n dimensions, and the elements in the first feature vector X correspond to the first-type state information, wherein the element of an i-th dimension is denoted as xi; the second feature vector Y includes elements of n dimensions, and the elements in the second feature vector Y correspond to the second-type state information, wherein the element of an i-th dimension is denoted as yi.

Calculating the similarity between the first feature vector and the second feature vector includes:
calculating the similarity γ(X,Y) between the first feature vector X and the second feature vector Y based on the following formula:

$$γ ( X , Y ) = \frac{n Σ {x}_{i} {y}_{i} - Σ {x}_{i} Σ {y}_{i}}{\sqrt{n Σ {x}_{i}^{2} - {( Σ {x}_{i} )}^{2}} * \sqrt{n Σ {y}_{i}^{2} - {( Σ {y}_{i} )}^{2}}}$$

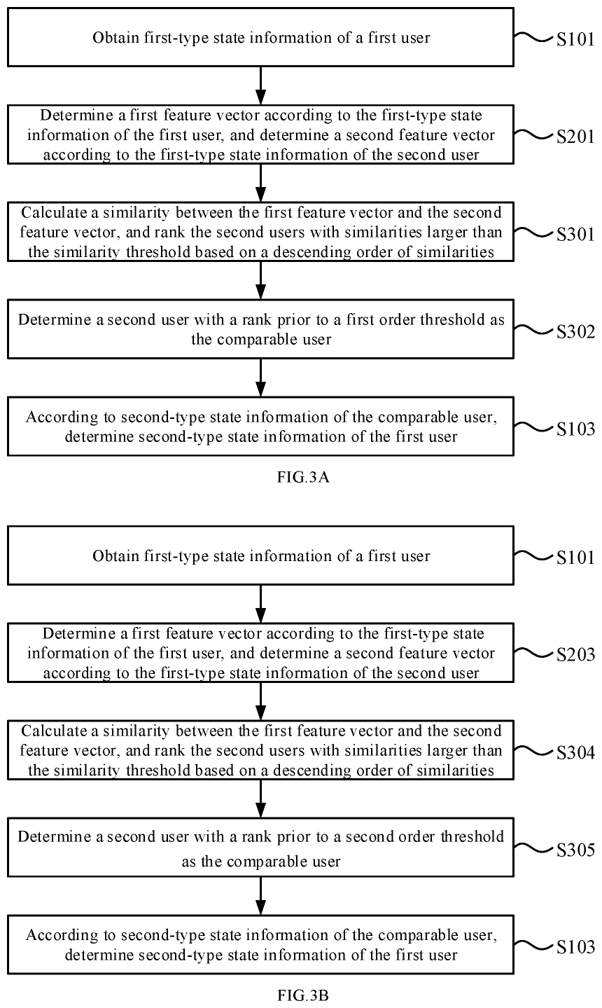

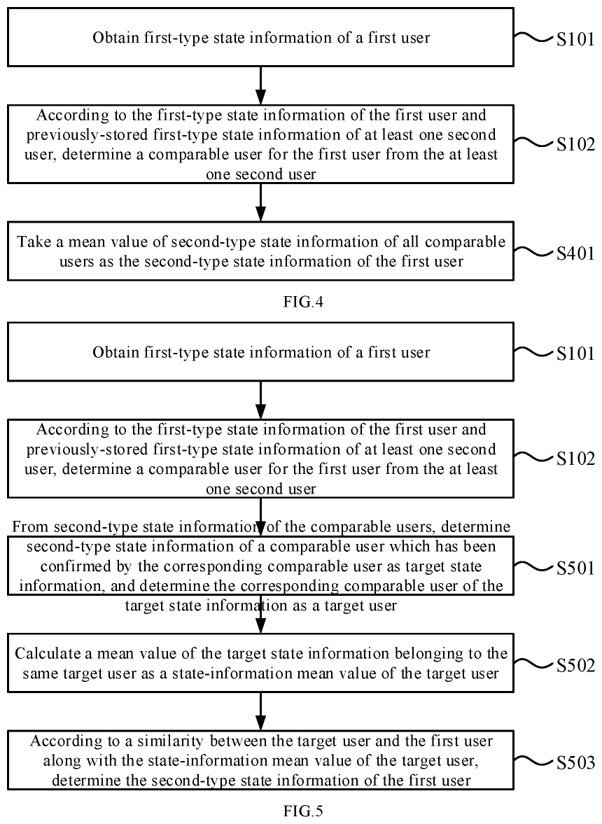

FIG. 3A is a flowchart illustrating a method of determining state information according to some embodiments of the present disclosure. As shown in FIG. 3A, determining a second user with a similarity larger than a similarity threshold as the comparable user includes the following steps.

At step S301, the second users with similarities larger than the similarity threshold are ranked based on a descending order of similarities.

At step S302, a second user with a rank prior to a first order threshold is determined as the comparable user.

In some embodiments, the first order threshold may be set as needed, which is not limited herein. For example, when the order threshold is set to 3, three second users with highest similarity may be determined as the comparable users.

In some embodiments, if the comparable user is determined only based on the relationship between the similarity and the similarity threshold, since a different number of comparable users may be determined from the second users based on a different first user, a larger number of comparable users may be determined for some first users, leading to a large amount of data volume, and bringing inconvenience to subsequent analysis and calculations.

In this embodiment, based on determination of similarity, the second users with similarities larger than the similarity threshold are ranked in a descending order of similarities, and the second users with a rank prior to the first order threshold are determined as comparable users. Therefore, a basically same number of comparable users may be determined for different first users, facilitating subsequent analysis and calculations.

For example, if 200 second users with similarities with the first user larger than the similarity threshold are determined, the 200 second users may be ranked in a descending order of similarities and the second users with ranked order located before 10 (the first order threshold may be set as needed), i.e. the first ten second users of the 200 second users, determined as comparable users. Thus, the scope of the comparable users is reduced, facilitating subsequent analysis and calculations.

FIG. 3B is a flowchart illustrating a method of determining state information according to some embodiments of the present disclosure. As shown in FIG. 3B, determining the comparable user for the first user from the at least one second user includes the following steps.

At step S303, a first feature vector is determined according to the first-type state information of the first user, and a second feature vector is determined according to the first-type state information of the second user.

At step S304, a similarity between the first feature vector and the second feature vector is calculated, and the second users are ranked in a descending order of similarities.

At step S305, a second user with a rank prior to a second order threshold is determined as the comparable user.

In some embodiments, the similarity between the first feature vector and the second feature vector is taken as a similarity between the first user and the second user, where implementation of determining similarity may be same as in the preceding embodiments and thus will not be repeated herein.

After the similarity is determined, the second users may be ranked in a descending order of similarities and further, the second users with a rank prior to the second order threshold are determined as comparable users. For example, if a similarity of 1000 second users with the first user is determined, the 1000 second users may be ranked in a descending order of similarities and the second users with a rank prior to 20 (the second order threshold may be set as needed), i.e. the first 20 second users of the 1000 second users, are determined as comparable users. Therefore, a same number of comparable users may be determined for different first users, facilitating subsequent analysis and calculations. In some embodiments, the second users may also be ranked in an ascending order of similarity, which will not be repeated herein.

FIG. 4 is a flowchart illustrating a method of determining state information according to some embodiments of the present disclosure. As shown in FIG. 4, according to the second-type state information of the comparable user, determining the second-type state information of the first user includes the following steps.

At step S401, a mean value of the second-type state information of all comparable users is calculated as the second-type state information of the first user.

In some embodiments, when a plurality of comparable users of the first user are determined, the mean value of the second-type state information of all comparable users may be taken as the second-type state information of the first user.

When the second-type state information is one piece of information, a mean value may be calculated for this piece of second-type state information. When the second-type state information includes a plurality of pieces of information, a mean value is calculated for each piece of second-type state information.

For example, if 10 comparable users with tare weights being 500 g, 550 g, 600 g, 650 g, 700 g, 750 g, 800 g, 860 g, 900 g and 950 g are determined, a mean value calculated is 725 g and the 725 g is provided as the second-type state information of the first user to the first user. FIG. 5 is a flowchart illustrating a method of determining state information according to some embodiments of the present disclosure. As shown in FIG. 5, according to the second-type state information of the comparable user, determining the second-type state information of the first user includes the following steps.

At step S501, from second-type state information of the comparable users, second-type state information of a comparable user which has been confirmed by the corresponding comparable user is determined as target state information, and the corresponding comparable user of the target state information is determined as a target user.

At step S502, a mean value of the target state information belonging to the same target user is calculated as a state-information mean value of the target user.

At step S503, according to a similarity between the target user and the first user along with the state-information mean value of the target user, the second-type state information of the first user is determined.

In some embodiments, the determined comparable user may be a first user which previously inputs the first-type state information to the terminal which determines the second-type state information based on the above some embodiments. That is, after determining the second-type state information of the first user based on the first-type state information of the first user, the terminal may store the first-type state information and the second-type state information of the first user, and when determining the second-type state information for a first user subsequently, take the first user of the previously-stored first-type state information and second-type state information as the second user so as to determine the second-type state information for subsequent first user.

For example, A user A uses the terminal, and based on the first-type state information of the user A and previously-stored first-type state information of at least one second user, the terminal determines a comparable user in the second user and determines the second-type state information of the user A based on the comparable user, and thus the second-type state information may be stored. Since the second-type state information of the user A is already stored, for a user B using the terminal subsequently, the user A already becomes a historical user, i.e. the second user. Hence, if A and B are same in the first user information, the A may be a comparable user of B, and further, the second user information of B may be determined based on the second user information of the A.

In this case, the second-type state information of the comparable user may also be determined by the terminal based on the above embodiments, but the second-type state information determined by the terminal may be inaccurate. After the second-type state information is pushed to the comparable user, it is to be determined whether the information is confirmed by the comparable user. When the comparable user confirms the information, it is determined that the second-type state information pushed to the comparable user is accurate. For the comparable user, the second-type state information of all comparable users may be inaccurate. Only when the second-type state information of the comparable user is confirmed by the comparable user can the second-type state information of the comparable user be accurate.

The operation performed by the comparable user to confirm the target state information may be performed on the terminal. For example, the terminal may display the target state information and a modification button and a confirmation button are disposed on the terminal. If the comparable user clicks the confirmation button, it may be determined based on a generated confirmation instruction that the comparable user confirms the target state information provided to the comparable user. If the comparable user clicks the modification button, the target state information may be adjusted based on a generated modification instruction. In this case, the comparable user does not confirm the target state information provided to the comparable user.

It is noted that, the operation performed by the comparable user to confirm the target state information may also be completed on a subsequent health detection device. For example, the terminal sends the target state information to the health detection device, and a modification button and a confirmation button are disposed on the terminal. If the comparable user clicks the confirmation button, it may be determined based on a generated confirmation instruction that the comparable user confirms the target state information provided to the comparable user. If the comparable user clicks the modification button, the target state information may be adjusted based on a generated modification instruction and then sent to the terminal. In this case, the comparable user does not confirm the target state information provided to the comparable user.

In some embodiments, after the second-type state information of the first user is determined based on the second-type state information of the comparable user, the second-type state information may be provided to the first user for confirmation/modification which is performed as before and thus will not be repeated herein.

Hence, from second-type state information of the comparable users, second-type state information of a comparable user which has been confirmed by the corresponding comparable user is determined as target state information, and the corresponding comparable user of the target state information is determined as a target user. That is, for the second-type state information of all comparable users, the second-type state information which has been confirmed by the comparable user may be determined as the target state information since such target state information is accurate.

In this case, since the comparable user is not a previously measured sample but a previous actual user, the comparable user may perform body composition analysis for a plurality of times. In this process, the comparable user will determine target state information for a plurality of times. Thus, for the comparable user, its second-type state information is a mean value of the plurality of pieces of target state information. Therefore, a mean value of the target state information belonging to the same target user may be calculated as a state-information mean value of the target user.

Then, when the second-type state information of the first user is determined, the second-type state information of the first user may be determined according to the similarity between the target user and the first user along with the state-information mean value of the target user. Since the state information of the target user is confirmed by the target user, it is accurate and thus the accuracy of determining the second-type state information of the second user may be guaranteed.

For example, the value v(a, p) of the second-type state information p of the first user a may be determined in the following formula:

$$v ( a , p ) = \frac{{Σ}_{b ∈ S ( a , I ) ⋂ N ( p )} {l}_{ab} \overline{{d}_{bp}}}{{Σ}_{b ∈ S ( a , U ) ⋂ N ( p )} {l}_{ab}}$$

where S(a,U) represents a set of comparable users U of a first user a, N(p) represents a set of target users, l_{ab} represents a similarity between the first user a and the target user b, and d̅_̅{̅b̅p̅}̅ represents the above state-information mean value of the target user b.

FIG. 6 is a flowchart illustrating a method of determining health index information according to some embodiments of the present disclosure. For example, in some embodiments, the method may be applied to a health detection device, for example, a composition analyzer and a blood glucose meter and the like. The health index information includes body composition information and blood glucose information and the like. When the health detection device is a composition analyzer, the device may perform body composition analysis. At this time, the health index information includes body composition information. The health detection device is in communication with the above terminal to receive the second-type state information determined by the above terminal. For example, the composition analyzer may be in communication with the terminal applicable to the above method of determining state information, to, for example, receive the second-type state information determined by the above terminal.

For example, in an application scenario of a health-care hut, a user logs on and inputs the first-type state information (body height, body weight and age and the like) through a terminal of a work station, and then the work station may calculate the second-type state information of the user (for example, de-tared information), and then sends the first-type state information and the second-type state information to a body composition analyzer, and the body composition analyzer performs body composition analysis based on the received first-type state information and second-type state information, and gives a result of the body composition analysis.

In use process, the user does not need to input the second-type state information on the body composition analyzer and the second-type state information transmitted by the work station may be directly used. Therefore, more conveniences will be brought to the user in operation, thus improving experiences of the user.

As shown in FIG. 6, with health index information as body composition information, the method may include the following steps.

At step S601, the body composition information of the first user is determined according to the first-type state information and the second-type state information of the first user in the method of determining state information in any of the above embodiments.

In some embodiments, the health detection device may perform body composition analysis for the first user according to the first-type state information and the second-type state information of the first user, so as to obtain the body composition information of the first user. The body composition information includes at least one of: fat ratio, moisture ratio and fatigue degree.

The first-type state information may be accurately determined by the first user and the second-type state information determined based on the above embodiment is also relatively accurate, and therefore, the body composition information may be accurately determined based on the first-type state information and the second-type state information.

In some embodiments, the method may further include providing the second-type state information of the first user to the first user.

In some embodiments, a screen may be disposed on the body composition analyzer to provide the second-type state information from the terminal of the above embodiment to the first user for viewing, confirming or modifying by the first user. For example, the body composition analyzer may prompt the second-type state information from the terminal of the above embodiment to the user in the form of voice broadcast, and the user may confirm or modify the second-type state information by voice control.

In some embodiments, the method further includes: when receiving a modification instruction for the second-type state information of the first user, determining the body composition information of the first user according to modified second-type state information of the first user.

The first user may confirm or adjust the second-type state information provided by the composition analyzer as needed. If the first user confirms the second-type state information, for example, clicks the confirmation button, the body composition information of the first user may be determined based on the first-type state information and the second-type state information provided to the first user; if the first user modifies the second-type state information, for example, clicks the modification button to input the second-type state information again, the body composition information of the first user may be determined based on the first-type state information and the modified second-type state information.

Hence, when the second-type state information of the first user is not accurately determined based on the second-type state information of the comparable user, the first user may modify the second-type state information based on its actual situations, helping improve the accuracy of calculating the body composition information.

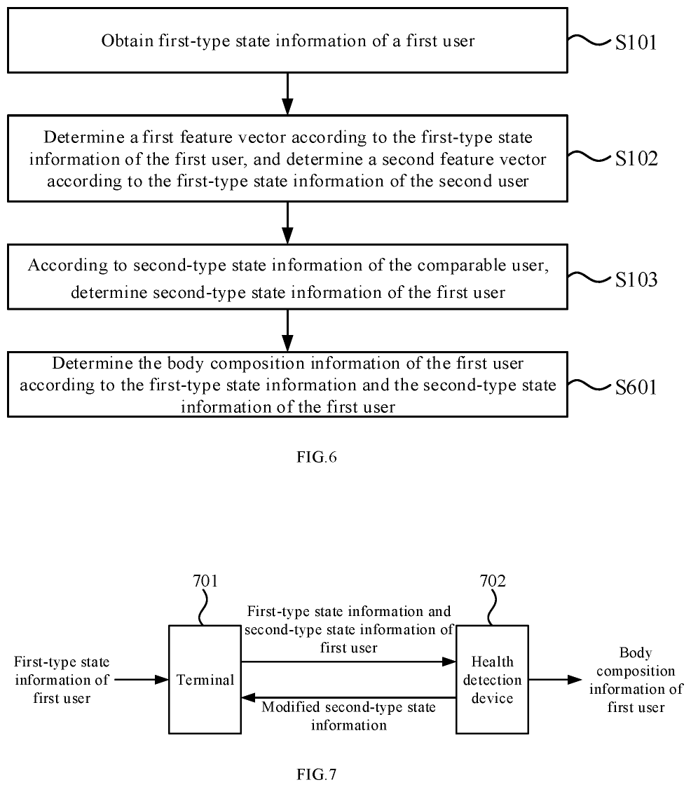

FIG. 7 is a schematic diagram illustrating a system for determining health index information according to some embodiments of the present disclosure. As shown in FIG. 7, the system may include a terminal 701 and a health detection device 702. The terminal may include but not limited to smart phone, tablet computer, personal computer, wearable device and work station and the like. The health detection device may include a body composition analyzer, a height and weight scale, a bone density tester and the like. The number of the health detection devices may be one or more, and the type and the number of the health detection devices are not limited herein.

The terminal is configured to: obtain first-type state information of a first user; according to the first-type state information of the first user and previously-stored first-type state information of at least one second user, determine a comparable user for the first user from the at least one second user; according to second-type state information of the comparable user, determine second-type state information of the first user; transmit the first-type state information and the second-type state information of the first user to the health detection device.

The health index information is health index information of the first user determined based on the first-type state information and the second-type state information of the first user.

In some embodiments, the first user may input the first-type state information to the terminal such that the terminal obtains the first-type state information of the first user. Implementation for the first user inputting the first-type state information includes but not limited to: touch input, voice input, direct measurement and the like. For example, the terminal is a work station of a health-care hut, and the first user may input the first-type state information through the work station.

The system may be disposed in an application scenario of the health-care hut. At least one work station and at least one type of detection devices are disposed in the hut and each type of detection devices may include a plurality of devices. The user may log onto the work station and input the first user information. The user may select a device to be used through the work station, and the work station may perform detection guidance to the user in the form of user interaction interface etc. The user may, according to the detection guidance provided by the work station, move to the position of the corresponding detection device for corresponding detection. After the user is detected by use of the detection device, the corresponding detection result will be sent to the work station for displaying. The health-care hut may be disposed in a place such as a community and residents in the community may go into the hut to perform health detection by a device, thus helping the residents to perform health self-management.

In some embodiments, the first-type state information may be state information which can be accurately determined by the user, for example, may include at least one of: gender information, age information, body height information, body weight information, position information, time information, season information, occupation information, people classification information (e.g. ordinary person, athlete). For example, the work station of the health-care hut provides a user interaction interface to display and prompt the user to input at least one of: gender information, age information, body height information, body weight information, occupation information, people classification information (e.g. ordinary person, athlete), and the user may perform input through the user interaction interface. For example, the work station of the health-care hut may obtain local geographical location information through automatic positioning and automatically obtain local time information and season information through the work station system. For example, part of the first-type state information may be stored after the first use of the user and will not be re-input in next use but automatically obtained based on login information. The user may also modify part of the first-type state information as needed and then store the modified information. For example, the invariable first-type state information such as gender will not be changed again after first input, and the first-type state information such as age which may change based on a specific rule may be updated based on the specific rule after the first input, whereas the information such as body weight and occupation etc. may change in subsequent use and thus may be modified as needed. In some embodiments, the second-type state information may be state information which generally cannot be determined by the user accurately, for example, may include at least one of tare weight information, blood pressure information, pulse information, and heart beat information. The tare weight information refers to a weight of other articles than the body of the user, which may include but not limited to clothing, shoes, mobile phone, and jewelry and the like.

In some embodiments, the terminal may pre-store first-type state information and second-type state information of at least one second user, for example, may pre-determine a large number of second users as samples, and then measures/inputs their first-type state information and second-type state information, thus obtaining accurate first-type state information and second-type state information of the second user.

In some embodiments, the first-type state information of the first user and the second-type state information determined by the first user may be stored as samples for subsequent use by the user.

In some embodiments, the first-type state information of the first user and the second-type state information determined by the first user may be subsequently added to previously-stored samples, so as to expand the samples. The second-type state information determined by the first user refers to the second-type state information confirmed by the first user after the second-type state information is determined for the first user based on the embodiment shown in FIG. 1 and pushed to the first user. This part of second-type state information is confirmed by the user and thus is accurate and may be stored as valid samples.

According to some embodiments of the present disclosure, after the first-type state information of the first user is obtained, a comparable user for the first user, for example, a second user with a similarity with the first user larger than a similarity threshold, may be determined from the second users according to the first-type state information of the second user and the first-type state information of the first user. If the first-type state information of the comparable user is similar to the state information of the first user, it is highly possible that the second-type state information of the comparable user is similar to the state information of the first user. Therefore, according to the second-type state information of the comparable user, the second-type state information of the first user may be determined.

Thus, it is not required for the first user to determine the second-type state information by subjective guess but determine a comparable user for the first user based on a large number of samples and further predict the second-type state information of the first user based on the second-type state information of the comparable user, thus facilitating improving the accuracy of determining the second-type state information. Further, the first-type state information and the second-type state information of the first user may be transmitted to the health detection device and thus it can be further guaranteed that the health index information of the first user can be accurately determined based on the first-type state information and the second-type state information.

In some embodiments, the terminal is configured to, according to information input by the first user, determine at least one of the following first-type state information of the first user: gender information, age information, body height information, body weight information, position information, time information, occupation information and people classification information.

In some embodiments, the second-type state information includes at least one of: tare weight information, blood pressure information, pulse information, and heart beat information.

In some embodiments, the health index information includes at least one of: fat ratio, moisture ratio and fatigue degree.

It is noted that, one or more comparable users may be determined. In a case of determining one comparable user, the second-type state information of the comparable user may be pushed as the second-type state information of the first user to the first user; in a case of determining a plurality of comparable users, the second-type state information of the first user may be determined based on the second-type state information of the plurality of comparable users, for example, with a similarity between the first user and the comparable user as a weight value, a weighted sum may be calculated for the second-type state information of the plurality of comparable users to obtain a result as the second-type state information of the first user. For another example, the second-type state information of the comparable user with highest similarity is taken as the second-type state information of the first user.

In some embodiments, the terminal is configured to determine a first feature vector according to the first-type state information of the first user, and determine a second feature vector according to the first-type state information of the second user; calculate a similarity between the first feature vector and the second feature vector; determine a comparable user for the first user from the at least one second user.

In some embodiments, in order to determine a similarity between the second user and the first user, a first feature vector may be determined according to the first-type state information of the first user, and a second feature vector may be determined according to the first-type state information of the second user, such that the first feature vector represents the first user and the second feature vector represents the second user. Further, a similarity between the first feature vector and the second feature vector may be calculated as a similarity between the first user and the second user, so as to determine a second user with a similarity larger than a similarity threshold as the comparable user.

In some embodiments, a similarity threshold may be set, such that a second user with a similarity larger than the similarity threshold may be determined as the comparable user. For example, the similarity threshold may be set as needed, which is not limited herein. For example, when the similarity threshold is set to 0.5, a second user with a similarity larger than 0.5 may be determined as the comparable user.

A number of pieces of the first-type state information of the first user may be n, and a number of pieces of the first-type state information of the second user may also be n. In this case, the first feature vector X may be a n-dimension vector including n pieces of first-type state information xi of the first user and correspondingly, the second feature vector Y may be a n-dimension vector including n pieces of first-type state information yi of the second user, where n is an integer equal to or larger than 1, and 1≤i≤n.

For example, there are five pieces of first-type state information: gender information, age information, body height information, body weight information, and time information.

The gender information may represent female with 0 and male with 1; the age information, the body height information, the body weight information may be corresponding values, for example, age 28 corresponds to the age information 28, body height 174 cm corresponds to the body height information 174, and body weight 50 kg corresponds to the body weight information 50; the time information may represent 6-9 o'clock with 1, and 9-12 o'clock with 2, 12-15 o'clock with 3, and 15-18 o'clock with 4.

For example, the first-type state information of the first user is female, age 25, height 165 cm and weight 50 kg, and a time that the first user inputs the first-type state information is 14 o'clock. In this case, the first feature vector generated based on these first-type state information may be represented as (0, 25, 167, 50, 14). Similarly, for the first-type state information of the second user, the second feature vector may also be generated as in the above implementation, which is not further described herein.

In some embodiments, the number of pieces of the first-type state information of the first user may be different from the number of pieces of the first-type state information of the second user as long as the similarity therebetween can be calculated. For example, if the number of pieces of the first-type state information of the first user is different from that of the second user, the missing value(s) may be supplemented as null to make the numbers of the first user and the second user to be the same, and the position of the corresponding vector element is set to 0, thus the first feature vector and the second feature vector may be of the same number of dimensions.

In some embodiments, the similarity between the first feature vector and the second feature vector may be determined by calculating an included angle between the first feature vector and the second feature vector. For example, a distance therebetween may be calculated by calculating their dot product or cosine value and the like, or determined in the following implementation. But no limitation is made to the two implementations and the specific implementation may be selected as needed.

In some embodiments, the first feature vector X includes elements of n dimensions, and the elements in the first feature vector X correspond to the first-type state information, wherein the element of an i-th dimension is denoted as xi; the second feature vector Y includes elements of n dimensions, and the elements in the second feature vector Y correspond to the second-type state information, wherein the element of an i-th dimension is denoted as yi, wherein n is an integer equal to or larger than 1 and 1≤i≤n.

The terminal is configured to calculate the similarity γ(X,Y) between the first feature vector X and the second feature vector Y based on the following formula:

$$γ ( X , Y ) = \frac{n Σ {x}_{i} {y}_{i} - Σ {x}_{i} Σ {y}_{i}}{\sqrt{n Σ {x}_{i}^{2} - {( Σ {x}_{i} )}^{2}} * \sqrt{n Σ {y}_{i}^{2} - {( Σ {y}_{i} )}^{2}}}$$

In some embodiments, the terminal is configured to rank the second users with similarities larger than the similarity threshold based on a descending order of similarities; and determine a second user with a rank prior to a first order threshold as the comparable user.

In some embodiments, the first order threshold may be set as needed, which is not limited herein. For example, when the order threshold is set to 3, three second users with highest similarity may be determined as the comparable users.

In some embodiments, if the comparable user is determined only based on the relationship between the similarity and the similarity threshold, since a different number of comparable users may be determined from the second users based on a different first user, a larger number of comparable users may be determined for some first users, leading to a large amount of data volume, and bringing inconvenience to subsequent analysis and calculations.

In this embodiment, based on determination of similarity, the second users with similarities larger than the similarity threshold are ranked in a descending order of similarities, and the second users with a rank prior to the first order threshold are determined as comparable users. Therefore, a basically same number of comparable users may be determined for different first users, facilitating subsequent analysis and calculations.

For example, if 200 second users with similarities with the first user larger than the similarity threshold are determined, the 200 second users may be ranked in a descending order of similarities and the second users with a rank prior to 10 (the first order threshold may be set as needed), i.e. the first ten second users of the 200 second users, determined as comparable users. Thus, the scope of the comparable users is reduced, facilitating subsequent analysis and calculations.

In some embodiments, the terminal is configured to: determine a first feature vector according to the first-type state information of the first user, and determine a second feature vector according to the first-type state information of the second user; rank the second users in a descending order of similarities; determine a second user with a rank prior to a second order threshold as the comparable user.

In some embodiments, the similarity between the first feature vector and the second feature vector is taken as a similarity between the first user and the second user, where the implementation of determining similarity may be same as in the preceding embodiments and thus will not be repeated herein.

After the similarity is determined, the second users may be ranked in a descending order of similarities and further, the second users with a rank prior to the second order threshold are determined as comparable users. For example, if a similarity of 1000 second users with the first user is determined, the 1000 second users may be ranked in a descending order of similarities and the second users with a rank prior to 20 (the second order threshold may be set as needed), i.e. the first 20 second users of the 1000 second users, are determined as comparable users. Therefore, a same number of comparable users may be determined for different first users, facilitating subsequent analysis and calculations.

In some embodiments, the terminal is configured to calculate a mean value of the second-type state information of all comparable users as the second-type state information of the first user.

In some embodiments, when a plurality of comparable users of the first user are determined, the mean value of the second-type state information of all comparable users may be taken as the second-type state information of the first user.

When the second-type state information is one piece of information, a mean value may be calculated for this piece of second-type state information. When the second-type state information includes a plurality of pieces of information, a mean value is calculated for each piece of second-type state information.

For example, if 10 comparable users with tare weights being 500 g, 550 g, 600 g, 650 g, 700 g, 750 g, 800 g, 860 g, 900 g and 950 g are determined, a mean value calculated is 725 g and the 725 g is provided as the second-type state information of the first user to the first user.

In some embodiments, the terminal is configured to: determine, from second-type state information of the comparable users, second-type state information of a comparable user which has been confirmed by the corresponding comparable user as target state information, and determine the corresponding comparable user of the target state information as a target user; calculate a mean value of the target state information belonging to the same target user as a state-information mean value of the target user; according to a similarity between the target user and the first user along with the state-information mean value of the target user, determine the second-type state information of the first user.

In some embodiments, the determined comparable user may be a first user which previously inputs the first-type state information to the terminal which determines the second-type state information based on the above some embodiments. That is, after determining the second-type state information of the first user based on the first-type state information of the first user, the terminal may store the first-type state information and the second-type state information of the first user, and when determining the second-type state information for a first user subsequently, take the first user of the previously-stored first-type state information and second-type state information as the second user so as to determine the second-type state information for subsequent first user.

For example, a user A uses the terminal, and based on the first-type state information of the user A and previously-stored first-type state information of at least one second user, the terminal determines a comparable user in the second user and determines the second-type state information of the user A based on the comparable user, and thus the second-type state information may be stored. Since the second-type state information of the user A is already stored, for a user B using the terminal subsequently, the user A already becomes a historical user, i.e. the second user. Hence, if A and B are same in the first user information, the A may be a comparable user of B, and further, the second user information of B may be determined based on the second user information of the A.

In this case, the second-type state information of the comparable user may also be determined by the terminal based on the above embodiments, but the second-type state information determined by the terminal may be inaccurate. After the second-type state information is pushed to the comparable user, it is to be determined whether the information is confirmed by the comparable user. When the comparable user confirms the information, it is determined that the second-type state information pushed to the comparable user is accurate. For the comparable user, the second-type state information of all comparable users may be inaccurate. Only when the second-type state information of the comparable user is confirmed by the comparable user can the second-type state information of the comparable user be accurate.

The operation performed by the comparable user to confirm the target state information may be performed on the terminal. For example, the terminal may display the target state information and a modification button and a confirmation button are disposed on the terminal. If the comparable user clicks the confirmation button, it may be determined based on a generated confirmation instruction that the comparable user confirms the target state information provided to the comparable user. If the comparable user clicks the modification button, the target state information may be adjusted based on a generated modification instruction. In this case, the comparable user does not confirm the target state information provided to the comparable user.

It is noted that, the operation performed by the comparable user to confirm the target state information may also be completed on a subsequent health detection device. For example, the terminal sends the target state information to the health detection device, and a modification button and a confirmation button are disposed on the terminal . If the comparable user clicks the confirmation button, it may be determined based on a generated confirmation instruction that the comparable user confirms the target state information provided to the comparable user. If the comparable user clicks the modification button, the target state information may be adjusted based on a generated modification instruction and then sent to the terminal. In this case, the comparable user does not confirm the target state information provided to the comparable user.

Hence, from second-type state information of the comparable users, second-type state information of a comparable user which has been confirmed by the corresponding comparable user may be determined as target state information, and the corresponding comparable user of the target state information may be determined as a target user. That is, for the second-type state information of all comparable users, the second-type state information which has been confirmed by the comparable user may be determined as the target state information since such target state information is accurate.

In this case, since the comparable user is not a previously measured sample but is an actual prior user, the comparable user may perform body composition analysis for a plurality of times. In this process, the comparable user will determine the target state information for a plurality of times. Thus, for the comparable user, its second-type state information is a mean value of a plurality of pieces of target state information. Therefore, a mean value of the target state information belonging to the same target user may be calculated as a state-information mean value of the target user.

Then, when the second-type state information of the first user is determined, the second-type state information of the first user may be determined according to the similarity between the target user and the first user along with the state-information mean value of the target user. Since the state information of the target user is confirmed by the target user, it is accurate and thus the accuracy of determining the second-type state information of the second user may be guaranteed.

For example, the value v(a, p) of the second-type state information p of the first user a may be determined in the following formula:

$$v ( a , p ) = \frac{{Σ}_{b ∈ S ( a , I ) ⋂ N ( p )} {l}_{ab} \overline{{d}_{bp}}}{{Σ}_{b ∈ S ( a , U ) ⋂ N ( p )} {l}_{ab}}$$

where S(a,U) represents a set of comparable users U of a first user a, N(p) represents a set of target users, l_{ab} represents a similarity between the first user a and the target user b, and d̅_̅{̅b̅p̅}̅ represents the above state-information mean value the target user b.

In some embodiments, the health detection device is further configured to: provide the second-type state information of the second user to the second user, and when receiving a confirmation instruction of the second user for the second-type state information of the second user, determine the second-type state information of the second user is the target state information confirmed by the second user. In some embodiments, a screen may be disposed on the health detection device (e.g. body composition analyzer) to provide (e.g. display) the second-type state information from the terminal of the above embodiment to the first user for viewing by the first user. The user may confirm or modify the second-type state information displayed for composition analysis (e.g. performing operation, or voice control or the like through a screen of the device), and when receiving a confirmation instruction of the second user for the second-type state information of the second user, determine the second-type state information of the second user is the target state information confirmed by the second user.

In some embodiments, the terminal is configured to: according to the target state information confirmed by the second user obtained from the health detection device, determine, from second-type state information of the comparable users, second-type state information of a comparable user which has been confirmed by the corresponding comparable user as target state information, and determine the corresponding comparable user of the target state information as a target user.

After the health detection device detects the second user confirms the second-type state information, the health detection device may record the confirmed second-type state information as the target state information, determine, from second-type state information of the comparable users, second-type state information of a comparable user which has been confirmed by the corresponding comparable user as target state information, and determine the corresponding comparable user of the target state information as a target user, and notify the target state information and the corresponding target user to the terminal for the terminal to determine the second-type state information of the first user according to the similarity between the target user and the first user along with the state-information mean value of the target user.

In some embodiments, the health detection device is further configured to: when receiving a modification instruction for the second-type state information of the first user, determine the second-type state information of the first user according to modified second-type state information of the first user.

In some embodiments, the user may confirm or modify the second-type state information displayed for composition analysis, and when receiving a confirmation instruction of the second user for the second-type state information of the second user, determine the second-type state information of the second user is the target state information confirmed by the second user.

In an application scenario of a health-care hut, in the related arts, the user inputs the first-type state information such as body height and body weight on a work station, and then the work station may send the information such as body height and body weight to the health detection device such as body composition analyzer. Then the user moves to the health detection device to input the second-type state information such as tare weight and at this time, the health detection device can start measuring. According to some embodiments of the present disclosure, the user inputs the first-type state information such as body height and body weight on a work station, and then the work station (or a server in communication with the work station) may determine the second-type state information of the user such as tare weight, and send the above information such as body height, body weight and tare weight together to the health detection device such as body composition analyzer. After the user moves to the health detection device, the user does not need to perform manual inputting but directly start measuring. With the system for determining health index information provided by the embodiments of the present disclosure, the user may more conveniently perform operations, thus improving the user experiences.

Corresponding to the above embodiments of the method of determining state information and the method of determining health index information, the present disclosure further provides embodiments of an apparatus for determining state information and an apparatus for determining health index information.

FIG. 8 is a block diagram illustrating an apparatus for determining state information according to some embodiments of the present disclosure. The apparatus shown in the embodiments is applicable to an electronic device such as a terminal provided for a user to input state information. The terminal includes but not limited to smart phone, tablet computer, personal computer, wearable device and the like.

As shown in FIG. 8, the apparatus for determining state information may include:
a state obtaining module 801, configured to obtain first-type state information of a first user;
a similarity determining module 802, configured to, according to the first-type state information of the first user and previously-stored first-type state information of at least one second user, determine a comparable user for the first user from the at least one second user;
a state determining module 803, configured to, according to second-type state information of the comparable user, determine second-type state information of the first user.

In some embodiments, the similarity determining module is configured to: determine a first feature vector according to the first-type state information of the first user, and determine a second feature vector according to the first-type state information of the second user; calculate a similarity between the first feature vector and the second feature vector, and determine a second user with a similarity larger than a similarity threshold as the comparable user.

In some embodiments, the first feature vector X includes elements of n dimensions, and the elements in the first feature vector X correspond to the first-type state information, wherein the element of an i-th dimension is denoted as xi; the second feature vector Y includes elements of n dimensions, and the elements in the second feature vector Y correspond to the second-type state information, wherein the element of an i-th dimension is denoted as yi, n is an integer equal to or larger than 1, 1≤i≤n.

The similarity determining module is configured to calculate the similarity γ(X,Y) between the first feature vector X and the second feature vector Y based on the following formula:

$$γ ( X , Y ) = \frac{n Σ {x}_{i} {y}_{i} - Σ {x}_{i} Σ {y}_{i}}{\sqrt{n Σ {x}_{i}^{2} - {( Σ {x}_{i} )}^{2}} * \sqrt{n Σ {y}_{i}^{2} - {( Σ {y}_{i} )}^{2}}}$$

In some embodiments, the similarity determining module is configured to: rank the second users with similarities larger than the similarity threshold based on a descending order of similarities; determine a second user with a rank prior to a first order threshold as the comparable user.

In some embodiments, the similarity determining module is configured to: determine a first feature vector according to the first-type state information of the first user, and determine a second feature vector according to the first-type state information of the second user; calculate a similarity between the first feature vector and the second feature vector, and rank the second users according to a descending order of similarities; determine a second user with a rank prior to a second order threshold as the comparable user.

In some embodiments, the state determining module is configured to: calculate a mean value of the second-type state information of all comparable users as the second-type state information of the first user.

In some embodiments, the state determining module is configured to: determine, from second-type state information of the comparable users, second-type state information of a comparable user which has been confirmed by the corresponding comparable user as target state information, and determine the corresponding comparable user of the target state information as a target user; calculate a mean value of the target state information belonging to the same target user as a state-information mean value of the target user; according to a similarity between the target user and the first user along with the state-information mean value of the target user, determine the second-type state information of the first user.

In some embodiments, the method of determining state information is applicable to a terminal.

In some embodiments, the first-type state information includes at least one of: gender information, age information, body height information, body weight information, position information, time information and occupation information.

In some embodiments, the second-type state information includes at least one of: tare weight information, blood pressure information, pulse information, and heart beat information.

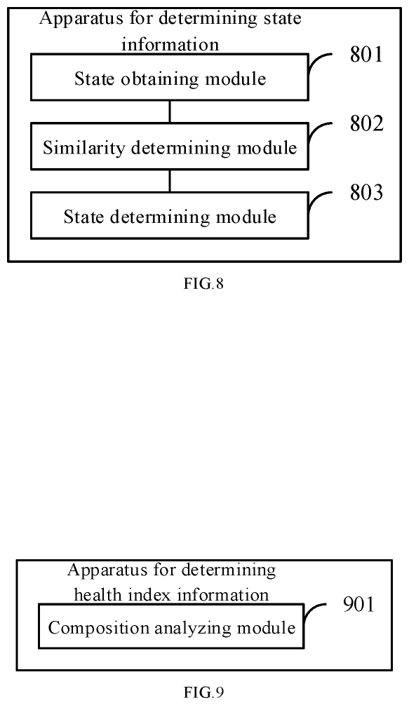

FIG. 9 is a block diagram illustrating an apparatus for determining health index information according to some embodiments of the present disclosure. For example, in some embodiments, the apparatus may be applied to a health detection device, for example, composition analyzer, blood glucose meter and the like. When the health detection device is a composition analyzer, the device may perform body composition analysis. At this time, the health index information includes body composition information. The health detection device is in communication with the above terminal to receive the second-type state information determined by the above terminal. For example, the composition analyzer may be in communication with the terminal applicable to the above method of determining state information, to, for example, receive the second-type state information determined by the above terminal.

As shown in FIG. 9, with health index information as body composition information, the apparatus for determining health index information includes:
a composition analyzing module 901, configured to, according to the first-type state information and the second-type state information of the first user in the method of determining state information according to any one of the above embodiments, determine the health index information of the first user.

In some embodiments, the method further includes: a state displaying module, configured to provide the second-type state information of the first user to the first user.

In some embodiments, the composition analyzing module is further configured to: when receiving a modification instruction for the second-type state information of the first user, determine the body composition information of the first user according to modified second-type state information of the first user.

In some embodiments, the apparatus for determining health index information is applicable to a health detection device.

In some embodiments, the body composition information includes at least one of: fat ratio, moisture ratio and fatigue degree.

The specific implementation of the various modules in the apparatus of the above embodiments perform operations is already elaborated in the embodiments of relevant methods and thus will not be repeated herein.

Since the apparatus embodiments substantially correspond to the method embodiments, a reference may be made to part of the descriptions of the method embodiments for the related part. The apparatus embodiments described above are merely illustrative, where the modules described as separate members may be or not be physically separated, and the members displayed as modules may be or not be physical units, i.e., may be located in one place, or may be distributed to a plurality of network units. Part or all of the modules may be selected according to actual requirements to implement the objectives of the solutions in the embodiments. Those of ordinary skill in the art may understand and carry out them without creative work.

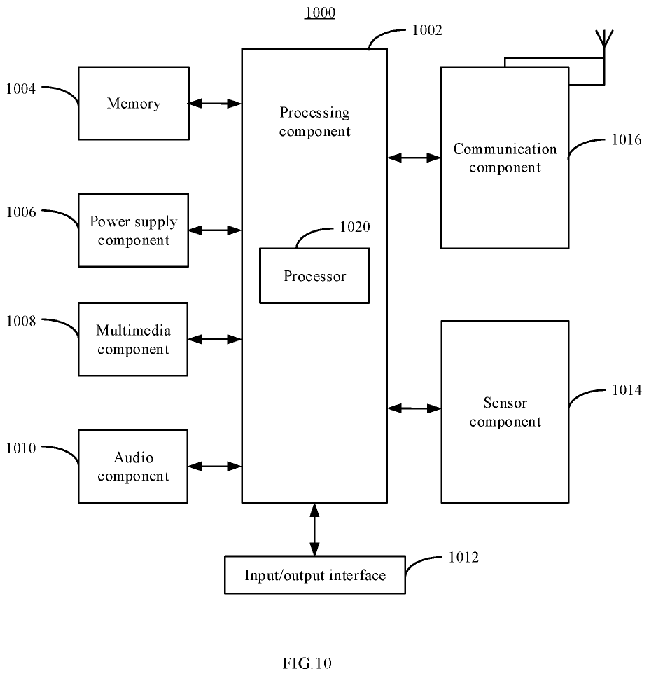

FIG. 10 is a block diagram of an apparatus 1000 according to some embodiments of the present disclosure. For example, the apparatus 1000 may be a mobile phone, a computer, a digital broadcast terminal, a message transceiver, a game console, a tablet device, a medical device, a fitness device, a personal digital assistant, and the like.

As shown in FIG. 10, the apparatus 1000 may include one or more of the following components: a processing component 1002, a memory 1004, a power supply component 1006, a multimedia component 1008, an audio component 1010, an input/output (I/O) interface 1012, a sensor component 1014 and a communication component 1016.

The processing component 1002 generally controls overall operations of the apparatus 1000, such as operations associated with display, phone calls, data communications, camera operations, and recording operations. The processing component 1002 may include one or more processors 1020 to execute instructions to complete all or part of the steps of the above methods. In addition, the processing component 1002 may include one or more modules which facilitate the interaction between the processing component 1002 and other components. For example, the processing component 1002 may include a multimedia module to facilitate the interaction between the multimedia component 1008 and the processing component 1002.

The memory 1004 is configured to store various types of data to support the operation of the apparatus 1000. Examples of such data include instructions for any application or method operated on the apparatus 1000, contact data, phonebook data, messages, pictures, videos, and so on. The memory 1004 may be implemented by any type of volatile or non-volatile storage devices or a combination thereof, such as a Static Random Access Memory (SRAM), an Electrically Erasable Programmable Read-Only Memory (EEPROM), an Erasable Programmable Read-Only Memory (EPROM), a Programmable Read-Only Memory (PROM), a Read-Only Memory (ROM), a magnetic memory, a flash memory, a magnetic or compact disk.

The power supply component 1006 supplies power for different components of the apparatus 1000. The power supply component 1006 may include a power supply management system, one or more power supplies, and other components associated with generating, managing and distributing power for the apparatus 1000.

The multimedia component 1008 includes a screen that provides an output interface between the apparatus 1000 and a user. In some examples, the screen may include a liquid crystal display (LCD) and a touch panel (TP). If the screen includes a touch panel, the screen may be implemented as a touch screen to receive input signals from the user. The touch panel includes one or more touch sensors to sense touches, slides, and gestures on the touch panel. The touch sensor may not only sense the boundary of touch or slide actions but also detect the duration and pressure associated with touch or slide operations. In some examples, the multimedia component 1008 includes a front camera and/or a rear camera. When the apparatus 1000 is in an operation mode, such as a shooting mode or a video mode, the front camera and/or the rear camera may receive external multimedia data. Each of the front and rear cameras may be a fixed optical lens system or have a focal length and an optical zoom capability.

The audio component 1010 is configured to output and/or input audio signals. For example, the audio component 1010 includes a microphone (MIC) configured to receive an external audio signal when the apparatus 1000 is in an operation mode, such as a call mode, a recording mode, and a voice recognition mode. The received audio signal may be further stored in the memory 1004 or transmitted via the communication component 1016. In some embodiments, the audio component 1010 also includes a loudspeaker for outputting an audio signal.

The I/O interface 1012 provides an interface between the processing component 1002 and a peripheral interface module which may be a keyboard, a click wheel, a button, or the like. These buttons may include, but are not limited to a home button, a volume button, a start button, and a lock button.

The sensor component 1014 includes one or more sensors for providing a status assessment in various aspects to the apparatus 1000. For example, the sensor component 1014 may detect an open/closed state of the apparatus 1000, and the relative positioning of components, for example, the component is a display and a keypad of the apparatus 1000. The sensor component 1014 may also detect a change in position of the apparatus 1000 or a component of the apparatus 1000, the presence or absence of a user in contact with the apparatus 1000, the orientation or acceleration/deceleration of the apparatus 1000 and a change in temperature of the apparatus 1000. The sensor component 1014 may include a proximity sensor configured to detect the presence of nearby objects without any physical contact. The sensor component 1014 may also include a light sensor, such as a CMOS or CCD image sensor, for use in imaging applications. In some examples, the sensor component 1014 may also include an acceleration sensor, a gyro sensor, a magnetic sensor, a pressure sensor, or a temperature sensor.

The communication component 1016 is configured to facilitate wired or wireless communication between the apparatus 1000 and other devices. The apparatus 1000 may access a wireless network based on a communication standard, such as WiFi, 2G or 3G, 4G LTE, 5G NR or a combination thereof. In an embodiment, the communication component 1016 receives broadcast signals or broadcast associated information from an external broadcast management system via a broadcast channel. In an embodiment, the communication component 1016 also includes a near field communication (NFC) module to facilitate short range communication. For example, the NFC module may be implemented based on a radio frequency identification (RFID) technology, an infrared data association (IrDA) technology, an ultrawideband (UWB) technology, a Bluetooth (BT) technology, and other technologies.

In an embodiment, the apparatus 1000 may be implemented by one or more application specific integrated circuits (ASICs), digital signal processors (DSPs), digital signal processing devices (DSPDs), programmable logic devices (PLDs), a field programmable gate array (FPGA), a controller, a microcontroller, a microprocessor or other electronic elements for performing the above method of determining state information.

In an embodiment, there is also provided a non-transitory computer readable storage medium including instructions, such as a memory 1004 including instructions, where the instructions are executable by the processor 1020 of the apparatus 1000 to perform the method of determining state information as described above. For example, the non-transitory computer readable storage medium may be a Read-Only Memory (ROM), a Random Access Memory (RAM), CD-ROM, a magnetic tape, a floppy disk, and an optical data storage device and so on.

Each component/module/unit of the present disclosure may be implemented by hardware, or by software module operated in one or more processors, or by a combination thereof.

It should be understood that although various steps in the flowchart of the accompanying drawings are displayed in a sequence as indicated by an arrow, these steps are not necessarily performed in the sequence indicated by the arrow. Unless otherwise clearly stated in the present disclosure, these steps are not limited to any strict sequence and may be performed in another sequence. Furthermore, at least part of the steps in the flowchart of the accompanying drawings may include a plurality of sub-steps or a plurality of stages. These sub-steps or stages are not necessarily completed at a same moment but may be performed at different moments. These sub-steps or stages are also not necessarily performed in sequence but may be performed in turns or alternately with at least part of other steps or the sub-steps or stages of other steps.

Other implementations of the present disclosure will be apparent to those skilled in the art from consideration of the specification and practice of the present disclosure herein. The present disclosure is intended to cover any variations, uses, modification or adaptations of the present disclosure that follow the general principles thereof and include common knowledge or conventional technical means in the related art that are not disclosed in the present disclosure. The specification and embodiments are considered as exemplary only, with a true scope and spirit of the present disclosure being indicated by the following claims.

It is to be understood that the present disclosure is not limited to the precise structure described above and shown in the accompanying drawings, and that various modifications and changes may be made without departing from the scope thereof. The scope of the present disclosure is limited only by the appended claims.

It shall be noted that the relational terms such as “first” and “second” used herein are merely intended to distinguish one entity or operation from another entity or operation rather than to require or imply any such actual relation or order existing between these entities or operations. Also, the term “including”, “containing” or any variation thereof is intended to encompass non-exclusive inclusion, so that a process, method, article or device including a series of elements includes not only those elements but also other elements not listed explicitly or those elements inherent to such a process, method, article or device. Without more limitations, an element defined by the statement “including a . . . ” shall not be precluded to include additional same elements present in a process, method, article or device including the elements.

The above are detailed descriptions of a method and an apparatus provided according to the embodiments of the present disclosure. Specific embodiments are used herein to set forth the principles and the implementing methods of the present disclosure, and the descriptions of the above embodiments are only meant to help understanding of the method and the core idea of the present disclosure. Meanwhile, those of ordinary skill in the art may make alterations to the specific embodiments and the scope of application in accordance with the idea of the present disclosure. In conclusion, the contents of the present specification shall not be interpreted as limiting to the present disclosure.

1. A method of determining state information, comprising:
obtaining first-type state information of a first user;
according to the first-type state information of the first user and previously-stored first-type state information of at least one second user, determining a comparable user for the first user from the at least one second user;
according to second-type state information of the comparable user, determining second-type state information of the first user.

2. The method of claim 1, wherein according to the first-type state information of the first user and the previously-stored first-type state information of at least one second user, determining the comparable user for the first user from the at least one second user comprises:
determining a first feature vector according to the first-type state information of the first user, and determining a second feature vector according to the first-type state information of the second user;
calculating a similarity between the first feature vector and the second feature vector, and determining a second user with a similarity larger than a similarity threshold as the comparable user.

3. The method of claim 2, wherein the first feature vector, denoted as X, comprises elements of n dimensions, and the elements in the first feature vector X correspond to the first-type state information, wherein the element of an i-th dimension is denoted as xi; the second feature vector, denoted as Y, comprises elements of n dimensions, and the elements in the second feature vector Y correspond to the second-type state information, wherein the element of an i-th dimension is denoted as yi, n is an integer equal to or larger than 1, 1≤i≤n;
calculating a similarity between the first feature vector and the second feature vector comprises:
calculating a similarity γ(X,Y) between the first feature vector X and the second feature vector Y based on the following formula:
$$γ ( X , Y ) = \frac{n Σ {x}_{i} {y}_{i} - Σ {x}_{i} Σ {y}_{i}}{\sqrt{n Σ {x}_{i}^{2} - {( Σ {x}_{i} )}^{2}} * \sqrt{n Σ {y}_{i}^{2} - {( Σ {y}_{i} )}^{2}}}$$
wherein, the first feature vector X comprises elements of n dimensions, and the second feature vector Y comprises elements of n dimensions, xᵢ is an element of an i-th dimension in the first feature vector X, yᵢ is an element of an i-th dimension in the second feature vector Y, n is an integer equal to or larger than 1, 1≤i≤n.

4. The method of claim 2, wherein determining the second user with the similarity larger than the similarity threshold as the comparable user comprises:
ranking second users with similarities larger than the similarity threshold based on a descending order of similarities;
determining a second user with a rank prior to a first order threshold as the comparable user.

5. The method of claim 1, wherein determining the comparable user for the first user from the at least one second user comprises:
determining a first feature vector according to the first-type state information of the first user, and determining a second feature vector according to the first-type state information of the second user;
calculating a similarity between the first feature vector and the second feature vector, and ranking the second users according to a descending order of similarities;
determining a second user with a rank prior to a second order threshold as the comparable user.

6. The method of claim 1, wherein according to the second-type state information of the comparable user, determining the second-type state information of the first user comprises:
calculating a mean value of the second-type state information of all comparable users as the second-type state information of the first user.

7. The method of claim 1, wherein according to the second-type state information of the comparable user, determining the second-type state information of the first user comprises:
determining, from second-type state information of the comparable users, second-type state information of a comparable user which has been confirmed by the corresponding comparable user as target state information, and determining the corresponding comparable user of the target state information as a target user;
calculating a mean value of the target state information belonging to the same target user as a state-information mean value of the target user;
according to a similarity between the target user and the first user along with the state-information mean value of the target user, determining the second-type state information of the first user.

8. The method of claim 7, wherein the second-type state information of the first user is determined based on the following formula:
$$v ( a , p ) = \frac{{Σ}_{b ∈ S ( a , I ) ⋂ N ( p )} {l}_{ab} \overline{{d}_{bp}}}{{Σ}_{b ∈ S ( a , U ) ⋂ N ( p )} {l}_{ab}}$$
where, S(a,U) represents a set of comparable users U of a first user a, N(p) represents a set of target users, l_{ab} represents a similarity between the first user a and a target user b, and d̅_̅{̅b̅p̅}̅ represents the state-information mean value of the target user b.

9. The method of claim 1, wherein the method of determining state information is applicable to a terminal.

10. The method of claim 1, wherein the first-type state information comprises at least one of:
gender information, age information, body height information, body weight information, position information, time information and occupation information.

11. The method of claim 1, wherein the second-type state information comprises at least one of:
tare weight information, blood pressure information, pulse information, and heart beat information.

12. A method of determining health index information, comprising:
determining health index information of the first user according to the first-type state information and the second-type state information of the first user in the method of determining state information according to claim 1.

13. The method of claim 12, further comprising:
providing the second-type state information of the first user to the first user.

14. The method of claim 13, further comprising:
when receiving a modification instruction for the second-type state information of the first user, determining the health index information of the first user according to modified second-type state information of the first user.

15. The method of claim 12, wherein the method is applicable to a health detection device.

16. A system for determining health index information, comprising a terminal and a health detection device; wherein,
the terminal is configured to: obtain first-type state information of a first user; according to the first-type state information of the first user and previously-stored first-type state information of at least one second user, determine a comparable user for the first user from the at least one second user; according to second-type state information of the comparable user, determine second-type state information of the first user; transmit the first-type state information and the second-type state information of the first user to the health detection device;
the health detection device is configured to: based on the first-type state information and the second-type state information of the first user, determine the health index information of the first user.

17. The system of claim 16, wherein the terminal is configured to determine, from second-type state information of the comparable users, second-type state information of a comparable user which has been confirmed by the corresponding comparable user as target state information, and determine the corresponding comparable user of the target state information as a target user; calculate a mean value of the target state information belonging to the same target user as a state-information mean value of the target user; according to a similarity between the target user and the first user along with the state-information mean value of the target user, determine the second-type state information of the first user.

18. The system of claim 17, wherein the health detection device is further configured to: provide the second-type state information of the second user to the second user, and when receiving a confirmation instruction of the second user for the second-type state information of the second user, determine the second-type state information of the second user is the target state information confirmed by the second user.

19. The system of claim 18, wherein the terminal is configured to: according to the target state information confirmed by the second user obtained from the health detection device, determine, from second-type state information of the comparable users, second-type state information of a comparable user which has been confirmed by the corresponding comparable user as target state information, and determine the corresponding comparable user of the target state information as a target user.

20. The system of claim 16, wherein the health detection device is further configured to provide the second-type state information of the first user to the first user.

21.-25. (canceled)
